(19) United States
(12) Patent Application Publication
(10) Pub. No.: US 2009/0018125 A1
(43) Pub. Date: Jan. 15, 2009

(54) Pharmaceutical Composition and Its Use

(75) Inventors: Ulrich Mittmann, Freiburg (DE); Jean-Pierre Sachetto, Arlesheim (CH)

Correspondence Address: BACON & THOMAS, PLLC, 625 SLATERS LANE, FOURTH FLOOR, ALEXANDRIA, VA, 22314-1176, US

(73) Assignee: Tillotts Pharma AG, Ziefen (CH)

(21) Appl. No.: 11/629,093

(22) PCT Filed: Jun. 15, 2005
(86) PCT No.: PCT/EP2005/006412

§ 371 Date: Mar. 13, 2008

(30) Foreign Application Priority Data
Jun. 18, 2004 (GB) 0413729.5

(51) Int. Cl. A61K 31/4985; A61K 31/538; A61P 29/00

(52) U.S. Cl. 514/229.8; 514/249

(57) ABSTRACT

Polyunsaturated fatty acid (“PUFA”) or a pharmacologically acceptable salt or derivative thereof (such as EPA and/or DHA) is used in combination with at least one of an immunosuppressive agent or an antineoplastic agent, said agent(s) having at least one amino acid residue, or a pharmacologically acceptable salt or derivative thereof (such as methotrexate or cyclosporin) in the treatment of conditions involving acutely or chronically inadequate immune response. Specific conditions that may be treated include chronic inflammatory diseases (e.g. Crohn's disease and ulcerative colitis) and tumour diseases (e.g. bowel cancer and prostate cancer). One advantage of preferred embodiments of the invention is that bioavailability of immunosuppressive or antineoplastic agents is increased.

The invention relates to the use of at least one polyunsaturated fatty acid (“PUFA”) or pharmacologically acceptable salts or derivatives thereof in conjunction with at least one of an immunosuppressive agent and an antineoplastic agent or pharmacologically acceptable salts or derivatives thereof to treat conditions involving acutely or chronically inadequate immune response such as inflammatory bowel disease (“IBD”), rheumatoid arthritis, Behcet's syndrome, psoriasis, prostate cancer or bowel cancer.

According to Martindale (“The Complete Drug Reference”; 32^{nd} ed.; 1999), IBD covers chronic non-specific inflammatory conditions of the gastro-intestinal (“GI”) tract. The two major forms of IBD are Crohn's disease and ulcerative colitis.

Crohn's disease is characterised by thickened areas of the GI wall, with inflammation extending through all layers, deep ulceration and fissuring of the mucosa, and the presence of granulomas. Affected areas may occur in any area of the GI tract, interspersed with areas of normal tissue.

Ulcerative colitis is confined to the colon and rectum. Inflammation is superficial but continuous over the affected area but granulomas are rare. In mild disease, the rectum alone may be affected (proctitis). In severe disease, ulceration is extensive and much of the mucosa may be lost with an increased risk of toxic dilation of the colon, a potentially life-threatening complication.

Whilst there are differences between Crohn's disease and ulcerative colitis, similar treatments may be used in respect of both conditions. Corticosteroids are used in the treatment of more severe active disease and aminosalicylate derivatives may be used in the treatment of milder active disease. In addition, immunosuppressant therapy has been used in the treatment of chronic active disease. For example, azathioprine (CAS No. 446-86-6; 6-(1-methyl-4-nitroimidazol-5-ylthio)purine) has been shown to be of benefit to patients with Crohn's disease, particularly if complicated by fistulas, and may be useful in refractory ulcerative colitis.

Rheumatoid arthritis is an inflammatory arthritis in which joints, usually including those of the hands and feet, are inflamed resulting in swelling, pain and often the destruction of joints. It is considered to be an autoimmune disease in which components of the immune system attack the soft tissue that lines the joints. The condition is treated using non-steroidal antiinflammatory drugs (“NSAIDs”) such as ibuprofen, corticosteroids such as prednisone and immunosupressive drugs such as methotrexate and infliximab.

Behçet's syndrome is a chronic relapsing inflammatory disorder that can product recurring painful mouth sores, skin blisters, genital sores and swollen joints. The eyes, blood vessels, nervous system and digestive tract may also become inflamed. It is believed to be an autoimmune disorder. The condition is treated using corticosteroids such as prednisone and immunosuppressants such as cyclosporin.

Psoriasis is a chronic, recurring disease that causes one or more raised, red patches that have silvery scales and a distinct border between the patch and normal skin. It occurs because of an abnormally high rate of growth of skin cells thought to be caused by a problem with the immune system. The condition has been treated in the past with phototherapy, with topical drugs such as corticosteroids and with oral drugs such as cyclosporin and methotrexate.

Prostate cancer is the most common cancer among men in the USA and the second most common cause of cancer death. Three forms of treatment are currently used to treat prostate cancer: surgery, radiation therapy and hormonal therapy.

Bowel cancer is very common in the USA and Western Europe. About 50% of large bowel tumours occur in the rectum and about 20% in the sigmoid colon. The first-line treatment for localised disease is surgery. Adjuvant therapy, usually based on fluorouracil, has been widely used. Studies indicate that prolonged infusion of fluorouracil may improve the results of adjuvant therapy over bolus administration. Another approach is to use biochemical modulators such as folinic acid or immunomodulators such as levamisole.

Methotrexate (CAS No. 59-05-2; 4-amino-4-deoxy-10-methylpteroyl-L-glutamic acid) is an antineoplastic agent which acts as an antimetabolite of folic acid. It has been extensively used, often in association with other antineoplastic agents, in the treatment of a variety of malignant diseases including tumours of the mouth and stomach. There is no reference in Martindale to the use of methotrexate in the treatment of cancers of the colon or the rectum. Instead, fluorouracil (5-fluoropyrimidine-2,4-(1H,3H)-dione) appears to be the antineoplastic agent of choice to treat such malignant neoplasms.

Methotrexate is also an immunosuppressant that has been used in the treatment of IBD. Given intramuscularly once a week in a dose of 25 mg, methotrexate improves symptoms and reduces corticosteroid requirements in chronic active Crohn's disease (Feagan et al.; N. Engl. J. Med. 1995; 332; 292-7). Low dose methotrexate has been used for the induction of remission and for its steroid sparing effect in refractory and corticosteroid-dependent Crohn's disease (Egan et al.; Mayo Clin. Proc. 1996; 71; 69-80). It is disclosed in this latter reference that adverse effects are fewer and relapse less common with intramuscular rather than oral administration of methotrexate.

Cyclosporin (CAS No. 59865-13-3; cyclo{-[4-(E)-but-2-enyl-N,4-dimethyl-L-threonyl]-L-homoalanyl-(N-methylglycyl)-(N-methyl-L-leucyl)-L-valyl-(N-methyl-L-leucyl)-L-alanyl-D-alanyl-(N-methyl-L-leucyl)-(N-methyl-L-leucyl)-(N-methyl-L-valyl)-}) is an immunosuppressant that has been used in the treatment of various diseases considered to have an autoimmune component. Cyclosporin has been tried with variable success as a second-line drug in IBD. Intravenous high dose cyclosporin has been found to be effective in refractory ulcerative colitis (Lichtiger et al.; N. Engl. J. Med. 1994; 330; 1841-5) and may be useful if given by enema (Sandborn et al.; Am. J. Gastroenterol. 1993; 88; 640-5). However, the benefit in Crohn's disease is less clear. Although intravenous therapy is reportedly useful in healing refractory fistulae, lower oral doses have produced disappointing results in adults and children with active Crohn's disease (see for example Feagan et al.; N. Engl. J. Med. 1994; 330; 1846-51). When administered orally, cyclosporin is usually administered as liquid filled capsules or as an oily suspension.

Dactinomycin (CAS No. 50-76-0; N,N′-(2-amino-4,6-dimethyl-3-oxo-3H-phenoxazine-1,9-diyldicarbonyl)-bis[threonyl-D-valylprolyl(N-methylglycyl)(N-methylvaline) 1.5-3.1-lactone) is an antineoplastic agent that has been used in the treatment of gestational trophoblastic tumours, and other solid tumours including brain tumours, Wilm's tumour and various sarcomas. It is also an immunosuppressive agent and is usually administered intravenously.

It is known that eicosapenta-5,8,11,14,17-enoic acid (“EPA”), docosahexa-4,7,10,13,16,19-enoic acid (“DHA”) and other PUFAs are of use in the treatment of IBD (see, for example, EP-A-0244832, EP-A-0289204, EP-A-0311091 and WO-A-93/21912).

EP-A-0825858 (Buser et al.; published 21 Nov. 1996) discloses an oral dosage form comprising, as an active principle, a PUFA either in free acid form or as a pharmaceutically acceptable salt thereof. The oral dosage form is coated with a time but not pH dependent release coating material which allows releases of the PUFA in the ileum. The oral dosage form is used in the treatment of IBD.

Zerouga et al (Anti-Cancer Drugs 2002; 13; 301-311) synthesised and characterised a lipophilic phosphatidylcholine derivative containing two anti-cancer agents, DHA and methotrexate, attached covalently respectively at the sn-1 and sn-2 positions of the phospholipid. Results showed that DHA and methotrexate inhibited proliferation of murine leukaemia cells in vitro and that there is potential synergism between DHA and methotrexate when delivered concurrently as individual agents and when linked together through a phosphatidylcholine moiety.

Ferguson (Proc. Annu. Meet. Am. Assoc. Cancer Res. 1995; 36; A1722) studied the cytotoxic and chemomodulative effect of gamma-linoleic acid (“GLA”) against a human squamous carcinoma line and multidrug-resistant and carboplatin-resistant variants. Results showed that cells pre-treated with GLA were more sensitive to exposure to vincristine and carboplatin than untreated cells. Depending on the concentration of GLA, toxicity of carboplatin was enhanced upwards of 50% in both the human squamous carcinoma and the carboplatin-resistant variant cell lines. In addition, GLA was seen to enhance vincristine toxicity by up to 40%.

JP-A-63258816 (Imayado et al.; published on 26 Oct. 1988) discloses an anti-cancer composition comprising an anti-cancer agent low selective toxicity (selected from vincristine, daunorubicin, VP-16 and cisplatin) and a highly unsaturated fatty acid (e.g. GLA, arachadonic acid or EPA) having high selective toxicity. The reference discloses that the composition can be used in conventional fashion in applications using the anti-cancer agents indicated. The reference exemplifies in vitro studies of the effect of a 0.5wt % ethanol solutions of various combinations of the anti-cancer agents with one of the highly unsaturated fatty acids. The fatty acids all had 99% purity.

JP-A-8092129 (Yazawa et al.; published on 9 Apr. 1996) discloses a therapeutic treatment of eye conditions caused by autoimmune diseases comprising an immunosuppressant and EPA and/or DHA. Examples of immunosuppressants disclosed include dexamethasone, cyclosporin A, rapamycin, FK506, mizoribine, cyclophosamide, azathioprine and methotrexate. In the only exemplified embodiment, two patients taking cyclosporin A and two other patients taking FK506 were given soft gelatine capsules that contained tuna oil having 6% of EPA and 25% of DHA. The dose was 2400 mg per day which was divided into three parts for administration.

WO-A-98/09621 (Scott et al.; published on 12 Mar. 1998) discloses a method of treating and preventing the side effects of anti-cancer chemotherapy using a PUFA with a carbon chain length of 14 to 26 and with 2 to 6 double bonds in the molecule in cis- or trans-configuration. Preferred PUFAs include EPA and DHA. It is disclosed that the treatment is particularly suitable to treat the side effects resulting from the use of methotrexate, 5-fluorouracil, cyclophosphamide, cisplatin, doxorubicin, taxol and vincristine. The PUFAs may be administered at the same time as the anti-cancer drugs or preferably both prior to and during therapy with the anti-cancer drugs themselves. The doses of the PUFAs may be from 1 mg to 100 g per day and the PUFAs may be administered in any suitable manner including orally in the form of, for example, capsules and tablets.

In the only embodiment exemplified in WO-A-98/09621 in which methotrexate is used, a woman with breast cancer was treated with the “CMF” regime (cyclophosphamide, methotrexate and 5-fluorouracil) one week after receiving a cumulative dose of 30 g of GLA intravenously as the lithium salt with continued treatment with 2 g/day of oral lithium GLA. The side effects from the anti-cancer treatment were reduced.

In the only embodiment exemplified in WO-A-98/09621 in which a condition of the GI tract is treated, a man with metastatic colon cancer was treated with 5-fluorouracil. For two weeks prior to the chemotherapy and during the whole of the chemotherapy course, the man also received 3 g/day of the pure triglyceride of EPA. Again, the side effects from the chemotherapy were reduced.

Suzuki et al (J. Pharm. Sci. 1998; 87(10); 1196-202) discloses enhanced colonic and rectal absorption of the peptide hormone, insulin, in rats using a fatty acid emulsion. The emulsion was administered directly to rat intestinal loops in situ. Saturated, mono-unsaturated and polyunsaturated fatty acids including EPA and DHA were tested and the results indicated that the level of absorption of insulin was increased in line with the level of unsaturation of the fatty acids.

Barichello et al (Int. J. Pharm. 1999; 183(2); 125-32) discloses the rectal administration of insulin in rats using a Pluronic F-127 gel formulation containing unsaturated fatty acids.

WO-A-03/92671 (Krishnan; published on 13 Nov. 2003) discloses compositions for inhibiting angiogenesis. The compositions include an alkyl-substituted fatty acid, optionally with an immunosuppressant such as cyclosporin. The reference discloses many conditions that involve angiogenesis including various cancers, Crohn's disease and ulcerative colitis. The reference exemplifies in vitro studies of the inhibition of proliferation of human umbilical vein endothelial cells (“HUVEC”) using alkyl-substituted fatty acids and cyclosporin.

There is a need for an improved treatment of conditions involving acutely or chronically inadequate immune response, such as IBD and bowel cancer. It is, therefore, an objective of the present invention to provide an improved treatment of such conditions.

It is an objective of preferred embodiments of the present invention to improve the oral bioavailability of immunosuppressive agents and antineoplastic agents and, in particular, methotrexate or cyclosporin.

It is also an objective of preferred embodiments of the present invention to provide a method of administration of immunosuppressive agents and antineoplastic agents which reduces the systemic side effects (including nausea and vomiting) normally associated with parenteral routes of administration.

According to the first aspect, there is provided use of PUFA or a pharmacologically acceptable salt or derivative thereof in the manufacture of a medicament comprising at least one of an immunosuppressive agent and an antineoplastic agent, said agent(s) having at least one amino acid residue, or a pharmacologically acceptable salt or derivative thereof for the treatment of conditions involving acutely or chronically inadequate immune response, particularly intestinal conditions.

The first aspect of the present invention also provides for use of polyunsaturated fatty acid (“PUFA”) or a pharmacologically acceptable salt or derivative thereof and at least one of an immunosuppressive agent and an antineoplastic agent, said agent(s) having at least one amino acid residue, or a pharmacologically acceptable salt or derivative thereof in the manufacture of a medicament for the treatment of said conditions.

The first aspect of the present invention further provides for use of at least one of an immunosuppressive agent and an antineoplastic agent, said agent(s) having at least one amino acid residue, or a pharmacologically acceptable salt or derivative thereof in the manufacture of a medicament comprising PUFA or a pharmacologically acceptable salt or derivative thereof for the treatment of said conditions.

Treatment of these conditions is provided by topical application of the active agent(s) to the intestinal mucosa for a local or systemic effect. Where the condition to be treated is an intestinal condition, the topically applied active agents have a local effect.

The expression “immunosuppressive agent” is intended to mean pharmacologically acceptable compounds that have the effect of suppressing immune response in the human or animal body. The expression “antineoplastic agent” is intended to mean pharmacologically acceptable compounds that are cytotoxic to neoplastic cells. These expressions would be readily appreciated by the skilled man.

One advantage of administering at least one of an immunosupressive agent and an antineoplastic agent together with PUFA is that the oral bioavailability of the agent(s) is usually increased thereby allowing lower doses of the agent(s) to be administered to treat conditions involving acutely or chronically inadequate immune response than would otherwise have had to have been administered parenterally. Undesirable subcutaneous and intravenous dosing of agent(s) such as methotrexate or cyclosporin is therefore avoided resulting in reduction or elimination of unwanted side effects associated with high oral doses or parenteral use of the agent(s).

Without wishing to be bound by any particular theory, the inventors believe that increased uptake of the agent(s) results not from a pharmacological effect but instead from a physical effect arising from the interaction of the PUFA with the agent(s). It is believed that the agent(s) may be “packed” into a layer of PUFA which merges with intestinal mucosa cells. The higher fluidity of PUFAs when compared to fatty acids having lower levels of unsaturation may therefore lead to improved delivery of the agent(s).

In preferred embodiments, there is at least some post-gastric release of PUFA and the agent(s). Usually, all or substantially all of the release is post-gastric. The location of release of the active agent(s) in the intestines can be targeted and depends on the condition to be treated.

Where conditions, e.g. chronic inflammatory or tumour diseases, are to be treated systemically, release preferably occurs initially in the jejunum and continues along the majority of the ileum. Increased bioavailability of the active agent(s) is observed along this section of the bowel. Usually, in these embodiments, release is complete before the terminal ileum. A similar release profile may be used for the topical treatment of intestinal conditions such as inflammatory conditions of the small intestine (e.g. small intestinal Crohn's disease and Behçet's syndrome) and tumours of the small intestine.

In other embodiments, release would start in the small intestine and continue down the large bowel. For example, ileo-colonic release of the active agent(s) is preferred for the topical treatment of colonic conditions, e.g. inflammatory conditions (e.g. ulcerative colitis) of the colon and colo-rectal carcinomas.

The agent(s) are believed to interact with PUFA in contact with the intestinal mucosa. PUFA assists absorption of the agent(s) into the cells of the intestinal wall resulting in increased topical cellular uptake of the agent(s) into the immune cells and tumour cells of the mucosa and gut wall. Topical administration of the agent(s) is typically achieved providing high concentration of both PUFA and agent(s) available at the gut wall immune cells and/or tumour cells which the inventors believe results in significant potentiation of the effects of the components.

PUFAs are known to have antineoplastic and immunosuppressive activity (see above). Thus, a further advantage of the present invention is that co-administration of the agent(s) with PUFAs results in synergistic enhancement of the antineoplastic and/or immunosuppressive effects of the agent(s).

Suitable PUFAs include omega-3, omega-6 and omega-9 PUFAs but, whichever PUFAs are used, they are preferably unsubstituted. Suitable examples include EPA, DHA and GLA. At least one PUFA preferably is EPA or DHA. In preferred embodiments, a mixture of PUFAs comprising EPA and DHA is used. In such embodiments, the total amount of EPA and DHA in the mixture is preferably at least about 60 wt % of the mixture. The mixture may be in the form of a concentrated fish oil product. In preferred embodiments, the mixture comprises from about 50 to about 60 wt %, preferably 55 wt %, EPA and from about 15 to about 25 wt %, preferably 20 wt %, DHA.

The or at least one PUFA is preferably in the form of the free acid. Alternatively, the or at least one PUFA may be in the form of a pharmacologically acceptable salt such as the lithium or sodium salt, a pharmacologically acceptable ester such as the ethyl ester or the triglyceride ester or a pharmacologically acceptable n-3 phospholipid.

The immunosuppressive agent or the antineoplastic agent preferably has between one and fifteen amino acid residues. Suitable amino-acid derived immunosuppressive agents include methotrexate, dactinomycin, cyclosporin and a monoclonal antibody such as infliximab, natalizumab, daclizumab or muromonab. Suitable amino acid derived antineoplastic agents include methotrexate and dactinomycin.

Other, non-amino acid-derived, agents may be used in conjunction with the present invention. Such agents may have complex chemical structures, e.g. alkaloids, or are of fungal or bacterial origin. Examples of other, non-amino acid-derived immunosuppressive agents include 6-mercaptopurine (“6-MP”), cyclophosphamide, mycophenolate, prednisolone, sirolimus, dexamethasone, rapamycin, FK506, mizoribine, azothioprine and tacrolimus. Examples of other, non-amino acid-derived antineoplastic agents include fluorouracil, bleomycin, etoposide, taxol, vincristine, doxorubicin, cisplatin, daunorubicin and VP-16.

The medicament may comprises at least one oral dosage form comprising a mixture of said PUFA or said salt or derivative thereof and at least one of said immunosuppressive agent and said antineoplastic agent, or said salt or derivative thereof. In such embodiments, either the immunosuppressive agent or the antineoplastic agent is co-administered simultaneously with the PUFA. In such embodiments, the PUFA is independent from, i.e. not conjugated with, the other agent(s). The mixture may consist essentially of the mixture or may further comprise a pharmacologically acceptable vehicle.

Alternatively, the medicament may comprises at least one first oral dosage form comprising said PUFA or said salt or derivative thereof and at least one second oral dosage form comprising at least one of said immunosuppressive agent and said antineoplastic agent, or said salt or derivative thereof. In such embodiments, either the immunosuppressive agent or the antineoplastic agent may be co-administered simultaneously or sequentially with the PUFA. The first or second oral dosage form may further comprise a pharmacologically acceptable vehicle.

A suitable condition to be treated may be a chronic inflammatory disease. For example, the chronic inflammatory disease may result from hyperactive and in part defective control of immune response. Examples of such conditions include IBD (e.g. Crohn's disease and ulcerative colitis), rheumatoid arthritis, Behçet's syndrome and psoriasis.

Another suitable condition to be treated may be a tumour disease. For example, the tumour disease may result from lack of immune recognition and response to abnormal cells. Examples of such conditions include bowel cancer and prostate cancer.

The present invention has particular application in the topical treatment of intestinal conditions.

The intestinal condition to be treated may be IBD, for example Crohn's disease or ulcerative colitis. In such embodiments, the agent used is usually an immunosuppressive agent such as methotrexate, dactinomycin, cyclosporin or a monoclonal antibody.

The intestinal condition to be treated may be bowel cancer. In such embodiments, the antineoplastic agent may be methotrexate or dactinomycin. The invention has particular application in the treatment of cancer of the colon and/or the rectum.

The first aspect of the present invention also provides for use of PUFA or a pharmacologically acceptable salt or derivative thereof to increase the systemic bioavailability and the local bioavailability in at least a portion of the intestines of at least one of an immunosuppressive agent and an antineoplastic agent, said agent(s) having at least one amino acid residue, in a medicament for the treatment of conditions involving acutely or chronically inadequate immune response.

According to a second aspect of the present invention, there is provided a method of treatment of conditions involving acutely or chronically inadequate immune response comprising administering simultaneously or sequentially PUFA or a pharmacologically acceptable salt or derivative thereof and at least one of an immunosuppressive agent and an antineoplastic agent, said agents having at least one amino acid residue, or a pharmacologically acceptable salt or derivative thereof. The agents are usually administered in therapeutically effective amounts as required to treat the specific condition in question. In embodiments where a PUFA derivative is used, the derivative is usually an ester or an n-3 phospholipid. The treatment may have any of the features described above.

As mentioned above, PUFA and the agent(s) may be co-administered in the same oral dosage form or in different oral dosage forms. Thus, the expression “oral dosage form” is intended to include embodiments in which PUFA and the agent(s) are co-administered in the same oral dosage form and embodiments in which PUFA and the agent(s) are administered in separate oral dosage forms. Suitable forms include capsules (such as hard or soft gelatin capsules) and tablets. In embodiments using gelatin capsules, the gelatin may be Type A gelatin or Type B gelatin with Type A gelatin being preferred. The source of collagen from which the gelatin is made may be porcine, bovine or piscine. Porcine gelatin is preferred, particularly in embodiments in which PUFA in free acid form is used as the level of unwanted interaction of the PUFA with the capsule wall is reduced when compared with capsules using other sources of gelatin thereby improving the stability and effective shelf life of the formulation.

According to a third aspect of the present invention, there is provided an oral dosage form comprising PUFA or a pharmacologically acceptable salt or derivative thereof and at least one of an immunosuppressive agent and an antineoplastic agent, said agents having at least one amino acid residue, or a pharmacologically acceptable salt or derivative thereof. Preferably, the oral dosage form is coated with a delayed release coating, for example an enteric coating, to delay release of PUFA and the agent(s) until after passage through the stomach.

PUFA may be co-administered in a different oral dosage form from the agent(s). A suitable form for the separate oral administration of PUFA is disclosed in EP-A-0825858 (see above), the disclosure of which is incorporated herein by reference.

According to a fourth aspect of the present invention, there is provided a suitable form for the separate oral administration of the agent(s) in which an oral dosage form comprises at least one of an immunosuppressive agent and an antineoplastic agent, said agent(s) having at least one amino acid residue, or a pharmacologically acceptable salt or derivative thereof, wherein the oral dosage form is coated with a delayed release coating, for example an enteric coating. Such an oral dosage form may be used in conjunction with a separate oral dosage form comprising PUFA such as that disclosed in EP-A-0825858, the disclosure of which is incorporated herein by reference.

The oral dosage form(s) used in the topical treatment of intestinal conditions preferably delay release of PUFA and the agent(s) until reaching the affected portion of the intestine. In such embodiments, the oral dosage forms may be coated with a coating that allows post-gastric release of the or each active component for topical administration of the or each active component to the intestinal mucosa. Suitable coatings delay initial release of the agent(s) in either a pH dependent manner or a pH independent manner.

The oral dosage form(s) may be coated with a pH dependent release coating material. The pH of the bowel steadily increases from about 6 to about 7.5 from the duodenum to the colon. Different polyacrylate-based coating materials have been developed which dissolve at different pH of the intestine thereby releasing active(s) from the coated dosage forms at different points along the bowel. Suitable enteric coating materials include Eudragit™ L, Eudragit™ S and Eudragit™ F (Röhm Pharma Polymers).

The coating may delay initial release of the agent(s) in a pH independent manner. For example, in preferred embodiments, the coating delays initial release of the agent(s) in a time but not pH dependent manner. The oral dosage form(s) may be coated with a time but not pH dependent release coating material. In such embodiments, the location of release may be varied according to the thickness of such a coating. For example, as the thickness of the coating increases, so the location of initial release moves further along the bowel. Thus, a relatively thinner coating of such a material may provide initial release in the small intestine, e.g. in the jejunum, whereas a relatively thicker coating may provide initial release in the terminal ileum of the colon.

In some embodiments, the thickness of the coating may be sufficient to delay initial release of the active agent(s) for an average period of about 30 to about 60 minutes. Such embodiments would be suitable for ileal release of the agent(s). In other embodiments, the thickness of the coating may be sufficient to delay initial release of the active agent(s) for an average period of about 60 to about 120 minutes and preferably for an average period of about 90 to 120 minutes. Such embodiments would be suitable for initial release of the active agent(s) in or around the terminal ileum or colon.

The time but not pH dependent release coating material may be a neutral polyacrylate material such as a poly(ethylacrylate-methylmethacrylate) material. An example of a suitable material includes Eudragit NE 30-D (Rohm Pharma GmbH) which has an average molecular weight of about 800,000 and is usually used to form a sustained release matrix.

Another suitable pH independent release coating is a coating which biodegrades in the colon under the action of bacterial enzymes. An example of a suitable coating is a coating made from ethyl cellulose and amylose which is pH independent and degrades under the actions of colonic bacterial enzymes releasing the agent(s) in the colon. Other polymers which work in the same way would also be suitable.

Release of the or each active component is preferably sustained along at least a portion of the intestine. Any suitable method of sustaining release of the active components known in the art may be used. However, if a soft gelatin capsule coated with a time but not pH dependent release coating material, especially Eudragit NE 30 D, is used then release of the active agent(s) is achieved in a microdrop-wise fashion along a section of the bowel. Such a sustained release profile is believed by the inventors to be unique.

Without wishing to be bound by any particular theory, the inventors believe that the coating swells and perforates to allow intestinal fluid to pass through the coating. When the fluid comes into contact with the gelatin, the capsule swells to the point where the integrity of the wall fails and allows the contents of the capsule to escape as microdrops through the perforations in the coating. The capsule continues to travel along the intestine thereby sustaining release of the capsule contents along a section of the bowel.

The oral dosage form according to the third or fourth aspect of the present invention is usually suitable for use in the treatment of the human or animal body by diagnosis or therapy.

In a preferred embodiment, the medicament comprises dual oral dosage forms. The first oral dosage form is a soft gelatin capsule containing either 400 mg or 800 mg of a pharmaceutical composition comprising about 55 wt % EPA and about 20 wt % DHA, both in free acid form. The capsule is made from Type A porcine gelatin and is coated with Eudragit NE 30 D. The second oral dosage form may be, for example, a 2.5 mg methotrexate tablet or a 25 mg cyclosporin soft gelatin capsule. The second oral dosage form is preferably coated with Eudragit NE 30 D.

According to a fifth aspect of the present invention, there is provided a pharmaceutical product comprising at least one first oral dosage form comprising PUFA or a pharmacologically acceptable salt or derivative thereof and at least one second oral dosage form comprising at least one of an immunosuppressive agent and an antineoplastic agent, said agent(s) having at least one amino acid residue, or a pharmacologically acceptable salt or derivative thereof. Preferably, at least one of the first and second oral dosage forms is coated in any of the manners described above.

The following is a description, by way of example only, of presently preferred embodiments of the present invention.

### EXAMPLE

### Dual Oral Dosage Form

### First Oral Dosage Form (PUFA)

Transparent soft gelatin capsules were each filled with 1000 mg of a fish oil concentrate containing at least 60% by weight DHA and EPA (Incromega 3F60; Croda Universal Ltd, UK). The filled gelatin capsules were film coated with Eudragit® NE 30-D to provide resistance for 30 to 60 minutes at pH 5.5 by spraying with a film coating composition (see below) at 35 ml/min using 0.8 bar pressure at 25° C. and air drying for at least 30 mins at 25° C.

The film coating composition (for 50,000 capsules) was prepared by slowly adding silicon anti-foam emulsion (0.36 mg), brown iron oxide (E 172; 3.00 mg), titanium dioxide (2.35 mg) and talc (10 mg) in succession to water (75 mg) and agitating for 1 to 2 hours to form a very fine dispersion. A 30% aqueous dispersion of a poly(ethyl-acrylate-methylmethacrylate) having an average molecular weight of about 800,000 (Eudragit® NE 30D; 60 mg) and added to polysorbate 80 (MO 55 F; 0.2 mg) in a little water and the resultant mixture agitated. Silicon anti-foam emulsion (2 or 3 drops) was added to destroy the resultant foam and the aforementioned dispersion was slowly added. The vessel was washed with water (25 mg) and the dispersion stirred for 30 minutes before being filtered (150 μm)

### Second Oral Dosage Form (Methotrexate)

At least one tablet comprising 2.5 mg methotrexate sodium and a pharmacologically acceptable vehicle.

It will be appreciated that the invention is not restricted to the details described above with reference to the preferred embodiments but that numerous modifications and variations can be made without departing from the spirit or scope of the invention as defined by the following claims.

1. (canceled)

2. The method as claimed in claim 13 wherein at least one PUFA is eicosapenta-5,8,11,14,17-enoic acid (“EPA”).

3. The method as claimed in claim 13 wherein at least one PUFA is docosahexa-4,7,10,13,16,19-enoic acid (“DHA”).

4. The method as claimed in claim 13 wherein at least one PUFA is in free acid form.

5. The method as claimed in claim 13 wherein said agent is selected from the group consisting of methotrexate (4-amino-4-deoxy-10-methylpteroyl-L-glutamic acid) and salts thereof and dactinomycin (N,N′-(2-amino-4,6-dimethyl-3-oxo-3H-phenoxazine-1,9-diyldicarbonyl)-bis[threonyl-D-valylprolyl(N-methylglycyl)(N-methylvaline) 1.5-3.1-lactone).

6. The method as claimed in claim 13 where the administering is by oral route of administration.

7. The method as claimed in claim 13 comprising administering at least one oral dosage form comprising a mixture of said PUFA or said salt or derivative thereof and at least one agent selected from the group consisting of methotrexate and dactinomycin, or said salt or derivative thereof.

8. The method as claimed in claim 13 comprising administering at least one first oral dosage form comprising said PUFA or said salt or derivative thereof and at least one second oral dosage form comprising at least one agent selected from the group consisting of methotrexate and dactinomycin, or said salt or derivative thereof with simultaneous or sequential administration.

9. The method as claimed in claim 13 wherein the condition is a chronic inflammatory disease.

10. The method as claimed in claim 13 wherein the condition is selected from inflammatory bowel disease (“IBD”); Crohn's disease; ulcerative colitis; rheumatoid arthritis; Behçet's syndrome; and psoriasis.

11. The method as claimed in claim 13 wherein the condition is bowel cancer.

12. The method as claimed in claim 13 wherein the condition is prostate cancer.

13. Method of treatment of a condition selected from the group consisting of chronic inflammatory conditions, bowel cancer and prostate cancer comprising administering simultaneously or sequentially PUFA or a pharmacologically acceptable salt or derivative thereof and at least one agent selected from the group consisting of methotrexate and dactinomycin, or a pharmacologically acceptable salt or derivative thereof.

14. An oral dosage form comprising PUFA or a pharmacologically acceptable salt or derivative thereof and at least one agent selected from the group consisting of methotrexate and dactinomycin, or a pharmacologically acceptable salt or derivative thereof.

15. An oral dosage form as claimed in claim 14 wherein the oral dosage form is coated with a coating that delays release of the active agents until after passage through the stomach.

16. An oral dosage form comprising at least one agent selected from the group consisting of methotrexate and dactinomycin, or a pharmacologically acceptable salt or derivative thereof, wherein the oral dosage form is coated with a time but not pH dependent release coating material that delays release of the active agents until after passage through the stomach.

17. An oral dosage form as claimed in claim 14 for use in the treatment of the human or animal body by diagnosis or therapy.

18. A pharmaceutical product comprising at least one first oral dosage form comprising PUFA or a pharmacologically acceptable salt or derivative thereof and at least one second oral dosage form comprising at least one agent selected from the group consisting of methotrexate and dactinomycin, or a pharmacologically acceptable salt or derivative thereof.

19. A pharmaceutical product as claimed in claim 18 wherein at least one of the first and second oral dosage forms is coated with a coating that delays release of the active agent(s) until after passage through the stomach.

20. A method for treating a condition selected from the group consisting of chronic inflammatory conditions comprising administering simultaneously or sequentially therapeutically effective amounts of PUFA or a pharmacologically acceptable salt or derivative thereof and at least one monoclonal antibody or a pharmacologically acceptable salt or derivative thereof.

21.-23. (canceled)
